(19) United States
(12) Patent Application Publication
(10) Pub. No.: US 2017/0247766 A1
(43) Pub. Date: Aug. 31, 2017

(54) LATE ER+ BREAST CANCER ONSET ASSESSMENT AND TREATMENT SELECTION

(71) Applicant: THE UNIVERSITY OF NOTRE DAME DU LAC, South Bend, IN (US)

(72) Inventors: Steven Buechler, Granger, IN (US); Sunil Badve, Indianapolis, IN (US)

(73) Assignee: The University of Notre Dame du Lac, South Bend, IN (US)

(21) Appl. No.: 15/506,713

(22) PCT Filed: Aug. 26, 2015
(86) PCT No.: PCT/US15/47052

§ 371 Date: Feb. 24, 2017

Related U.S. Application Data
(60) Provisional application No. 62/041,750, filed on Aug. 26, 2014.

(51) Int. Cl. C12Q 1/68; G06F 19/18

(52) U.S. Cl. CPC C12Q 1/6886; G06F 19/18; C12Q 2600/118; C12Q 2600/158

(57) ABSTRACT

A method for determining the likelihood of late ER− breast cancer disease relapse/recurrence is disclosed. Late ER+ breast cancer disease onset and/or recurrence is determined for a period of 5 to 20 years after an initial ER+ breast cancer disease onset in a patient. An ER+ breast cancer patient is assigned a risk score that is compared to a defined threshold value, and identifies the risk score as low risk or high risk for late breast cancer recurrence. A late ER+ breast cancer gene panel of 8 to 15 genes is provided. Subjects having a risk score greater than or equal to that of the threshold value are at a relatively high risk of recurrent disease, and are determined to benefit from aggressive therapeutic intervention, whereas subjects having a risk score less than the threshold value are at a relatively low risk of recurrent disease, and could forego treatment.

### CROSS-REFERENCE TO RELATED APPLICATIONS

The present application claims the benefit under 35 U.S.C. 119(e) of the filing date of U.S. Application Ser. No. 62/041,750, filed on Aug. 26, 2014.

### BACKGROUND

Field

The present invention relates to the field of late ER+ breast cancer disease onset in humans, and methods for assessing risk of factors associated with the disease. The invention also relates to the field of methods for assessing appropriate treatment strategies for a patient with late ER+ breast cancer onset.

Description of Related Art

According to the American Cancer Society, more than one million people in the United States get cancer each year. If left untreated, cancer can be fatal.

When DNA in a cell is mutated and/or otherwise altered, the mutated and/or altered DNA is either repaired, or the cell dies. However, in some instances, such as in cells that manifest in the body as cancer, the cells containing mutated and/or altered DNA will replicate, and cancer disease in the body will progress. Cancer cells have genetic profiles of gene expression that are very different from native, non-mutated and/or non-altered cells, and for some cancers, the genetic profiles may continue to change unpredictably over time.

Cancer is a very dynamic, changing disease, and for this reason, among others, effective cancer treatment strategies must be tailored uniquely to the particular type of cancer and stage of cancer being treated in a particular patient. Cancers are typically characterized by the tissue in which they arise. For example, breast cancers arise in breast tissue, and particularly in epithelial cells of the breast tissue. Breast cancers can also be characterized by the presence of specific proteins, or protein variants on or in the cancer cells. Breast cancer cells may contain receptors that bind hormones that encourage cell growth such as estrogen or progesterone. Breast cancer cells that contain one or both of these hormone binding receptors are classified as hormone receptor-positive. Almost 67% of breast cancers are estrogen receptor positive (ER+) and/or progesterone receptor positive (PR+). About 20% of breast cancers express a different growth-promoting receptor called HER2/neu, and are referred to as HER2-positive. Cancer cells that lack all three of these receptors are classified as triple negative cancer.

The most invasive ER+ breast cancers are also categorized by the expression of hormone receptors and the amount of HER2, and the particular category of the cancer directly affects the treatment plan recommended for patients. HER2-positive breast cancers are known to grow and metastasize more aggressively than other types of breast cancers. Hormone receptor-negative breast cancers grow faster and do not respond to hormone treatment. Hormone receptor-positive breast cancers can be treated with hormone therapy drugs that lower estrogen levels or block receptors all together. While the outlook for women with ER+ breast cancers is statistically improved in the short-term, even these types ER+ cancers tend to recur years after treatment.

Gene expression patterns of one or multiple genes in a particular disease state, have been described for use in classifying breast cancers and are used as a tool in tailoring treatment options for an individual breast cancer patient. In addition to altered expression of estrogen receptors, progesterone receptors, and HER2/neu, altered expression of other genes are known to occur in breast cancer. For example, genes such as ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1, and ADAM17 have all been implemented in breast cancer cell gene pattern assessment. Altered expression of these genes and others have been associated with cancer in general, and the list of possibly relevant genes to breast cancer disease progression and onset continues to change as information concerning the disease progresses. Thus, the search goes on to identify the most powerful and diagnostic group of genes and/or genetic indicators for breast cancer and its various fauns.

Adjuvant chemotherapy and endocrine therapy have significantly improved breast cancer survival rates. However a significant number of women die from the recurrence of the disease long after onset and early treatment. The annual rate of ER breast cancer recurrence is at least 2% after fifteen years. Thus, identifying patients at a high risk of ER+ recurrence breast cancer is essential to devising treatment plans for a particular patient. Genomic signatures such as those used as the basis for Oncotype DX, Mammaprint, Breast Cancer Index (BCI), and Prosigna (PAM50 ROR) have been used to predict the risk of early ER+ breast cancer occurrence and recurrence. PAM50 ROR and BCI are described as predictive of ER+ breast cancer recurrence at between five and ten years after an initial ER+ breast cancer detection. These cases are described as “late recurrence.” Oncotype Dx has been reported to predict survival at the ten year mark after initial post ER+ breast cancer occurrence. The prognostic significance of Oncotype DX and PAM50 ROR has been observed to decrease after eight years.

Some diagnostic tests relating to outcomes of ER+ breast cancer relapse have been shown to lose prognostic ability after the five-year mark. Many of these diagnostic tests measure gene expression related to cell proliferation and cell cycle regulation, events that are associated with early relapse of breast cancer. Current tests based on prior identified genomic signatures improve upon what is already known in the field, by simply increasing the sensitivity of the tests or decreasing the number of patients for whom a prediction cannot be made. However, it has been observed that even these measures to improve patient disease treatment and outcome fail to consider and accommodate the dynamic cellular and genetic changes that occur in a patient years after initial breast cancer disease detection and treatment.

A need continues to exist in the medical art for improved methods for detecting and treating late ER+ breast cancer recurrence. Ideally, this medical need will be met with a genetic and/or cellular technique that captures the unique and more dynamic cellular and/or genetic events correlated with late ER+ breast cancer recurrence.

### SUMMARY

In a general and overall sense, the present compositions and methods satisfy these and other needs in the medical arts.

In one aspect, an assessment tool for late ER+ breast cancer recurrence in an “at risk” human ER+ breast cancer patient is provided. In one embodiment, the assessment tool comprises a threshold value that defines a reference heterogeneous late ER+ breast cancer marker of heterogeneous late ER+ breast cancer survivor population gene panel levels, wherein the assessment tool partitions an at risk human ER+ breast cancer tissue score into a high risk or a low risk ER+ breast cancer recurrence group.

As described herein, the heterogeneous late ER+ breast cancer survivor population gene panel comprises at least 8 genes selected from the group consisting of: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1 and ADAM17.

Employing those values and scores described as part of the various methods herein, a low risk human ER+ breast cancer tissue score below an about 60^{th} percentile of the score values in a heterogeneous ER+ breast cancer population indicates a patient with a statistically lower probability of developing late ER+ breast cancer recurrence from 5 to 20 years after an initial ER+ breast cancer occurrence, wherein a high risk human ER+ breast cancer tissue score at least above an about 60^{th} percentile or higher of the threshold score values in a heterogeneous ER+ breast cancer population indicates a patient with a statistically higher probability of developing late ER+ breast cancer recurrence from 5 to 20 years after an initial ER+ breast cancer occurrence. The level of each gene in the heterogeneous late ER+ breast cancer survivor population gene panel is identified with a cDNA, mRNA, cRNA or other nucleotide that is specific for the gene.

The group of genes highly correlated with late ER+ recurrence have an expression pattern identified as a bimodal expression pattern characteristic of tumors from a heterogeneous population of ER+ breast cancer survivors. A breast tumor tissue from a particular patient having had an ER+ breast cancer disease is assessed for late ER+ recurrence based on tissue expression levels of a late ER+ gene panel. Gene levels of each of genes in the late ER+ gene panel were measured in breast cancer tissue from a heterogeneous population of ER+ breast cancer survivors that did not develop a recurrence of ER+ breast cancer for at least 5 years, and to whom no therapeutic intervention was given. This provides a score value whereby a particular patient may be assessed as having a higher or a lower late-ER+ breast cancer recurrence risk.

Genes that have a generally bimodal expression in cancer patients are referred to herein as multi-state genes. According to the method, a panel of genes was determined to each constitute a multi-state gene for late ER+ breast cancer recurrence.

According to the method, eight or more of the late ER+ breast cancer multistate genes may be selected, and the expression levels for each of those genes assayed in an at-risk patient breast tissue sample in order to assess a patient prognosis. For example, the expression level of eight, nine, ten, eleven, twelve, thirteen, fourteen, fifteen, sixteen, seventeen, eighteen, nineteen, twenty, thirty, or forty late ER+ breast cancer multi-state genes may be ascertained according to embodiments of the invention to assess prognosis. The prognosis is based on comparing the patient's expression levels of the specific genes identified to that of a threshold score determined with expression levels of the same group of genes in a group of patient tissue samples from late ER+ breast cancer survivors that did not suffer recurrence of this cancer for at least 5 to 20 years and did not receive any subsequent therapeutic intervention. The threshold score provides a tool whereby those patients having a higher score have a poor prognosis, and those patients having expression levels below the threshold have a good prognosis.

In another aspect, a method for determining patient risk for late ER+ breast cancer recurrence is provided, comprising measuring a patient breast cancer tissue sample from an at risk ER+ breast cancer patient for levels of a heterogeneous late ER+ breast cancer survivor population gene panel comprising at least 8 genes, calculating a patient gene risk score between 0 and 1 for each gene of the gene panel measured in the patient breast cancer tissue sample, calculating a patient cumulative cancer test score between 0 to 100 from the patient gene risk score values for each gene of the gene panel; and comparing said patient cumulative cancer test score to a reference heterogeneous ER+ breast cancer population threshold value; wherein a patient cumulative cancer test score below about a 60^{th} percentile of the score values in a heterogeneous ER+ breast cancer population indicates a patient with a statistically lower probability of developing late ER+ breast cancer recurrence from 5 to 20 years after an initial ER+ breast cancer occurrence; and wherein a patient cumulative cancer test score at least above about a 60^{th} percentile or higher of the score values in a heterogeneous ER+ breast cancer population indicates a patient with a statistically higher probability of developing late ER+breast cancer recurrence from 5 to 20 years after an initial ER+ breast cancer occurrence. The patient breast tissue sample may comprise a frozen tissue, formalin fixed, paraffin embedded (FFPE) tissue, or a fresh tissue sample, and the levels of the heterogeneous late ER+ breast cancer survivor population gene panel may be provided by measure of a cDNA or cRNA prepared from the patient breast tissue sample. An ER+ breast cancer patient having a higher probability of late ER+ breast cancer recurrence is administered an aggressive anti-cancer therapeutic treatment, and an ER+ breast cancer patient having a lower probability of late ER+ breast cancer recurrence is not administered an aggressive anti-cancer therapeutic treatment.

In yet another aspect, a method for selecting a treatment regimen for an at risk late ER+ breast cancer recurrence patient is provided, comprising measuring a patient breast cancer tissue sample from an at risk ER+ breast cancer patient for levels of a heterogeneous late ER+ breast cancer survivor population gene panel comprising at least 8 genes, calculating a patient gene risk score between 0 and 1 for each gene of the gene panel measured in the patient breast cancer tissue sample, calculating a patient cumulative cancer test score between 0 to 100 from the patient gene risk score values for each gene of the gene panel; and comparing said patient cumulative cancer test score to a reference heterogeneous ER+ breast cancer population threshold value, and administering an aggressive anti-cancer therapeutic regimen to an ER+ breast cancer patient having a cumulative cancer test score at least within an about 60^{th} percentile or higher of the score values of a reference heterogeneous ER+ breast cancer population, or not administering an aggressive anti-cancer therapeutic regimen to an ER+ breast cancer patient not demonstrating a cumulative cancer test score at least above an about 60^{th} percentile or higher of the score values of a reference heterogeneous ER+ breast cancer population

The inclusion of a step for normalizing a patient sample gene measurement score against common endogenous genes decreases the genetic “noise” from nonspecific gene expression, thus enhancing the detectability of patient variation in the screening protocol. In addition, and because the normalized gene expression values for a reference population of patients range continuously from low values to high values with a large number of samples with values at a moderate level, and there are more relapse cases with high expression levels than low expression levels, and many more with moderate values that are as close to low (good prognosis) values as high (poor prognosis) values, additional steps are provided as part of the claimed protocols and screening techniques to reduce this uncertainty, or incidence of non-conclusive reading results, in patient sample readings.

Specifically, and in some embodiments of the methods/screening techniques, a gene risk score is determined for each gene/biomarker measured in the panel. In this process, a gene risk score is associated with each gene from 0 to 1, such that the gene risk scores increases along with the expression value of a gene/biomarker. A high risk patient sample would therefore have a gene risk score near 1, while a low risk patient sample would have a risk score near 0. Using this technique, there are very few samples (<10%) with values between 0.25 and 0.75, and very few patient samples with a moderate risk score. Thus, the use of risk scores, rather than expression values, in calculating a final test score minimizes the number of samples who receive a test score with an unclear prognosis. Thus, the precision and specificity of the screening and prognostic methods described here are significantly improved. Use of the risk scores also reduces the test's standard error, and increases the reliability of the test. As an even further improvement, the present screening and prognostic methods include yet another analysis to improve accuracy and precision in the use of a cancer test score to be identified for each patient. In this step, a cancer test score is calculated for each ER+ breast cancer patient, this cancer test score being a value of 1 to 100. This patient value, when compared to the values obtained from a heterogeneous population of ER+ breast cancer patients in a given population, is demonstrated by the present inventors to provide yet an additional added measure of predictive value of risk for cancer relapse to the present screening methods. Specifically, it was found that a patient having a cumulative cancer test score (determined according the methods described herein) that fell within an about 60^{th} percentile (or 65^{th}, 70^{th}, 80^{th}, or 60^{th} to 90^{th} percentile) or higher of a reference cumulative average cancer test score from a heterogeneous ER+ breast cancer population, could more reliably be identified as a patient at relatively much higher risk of relapse. Conversely, it was found that a patient having a cumulative cancer test score (determined according to the methods described herein) that did not fall within an about 60^{th} percentile (or 65^{th}, 70^{th}, 80^{th} , or 60^{th} to 90^{th} percentile) or higher of the reference cumulative average cancer test scores from a heterogeneous ER+ breast cancer population, could more reliably be identified as a patient at a relatively much lower risk of relapse. The lower range of the percentile may also be described as the lower 20^{th}, 30^{th}, 40th, 50^{th}, or less than 60^{th} percentile, of the reference cumulative average cancer test scores from a heterogeneous ER+ breast cancer population, and is correlated with relatively low risk cancer relapse ER+ patients.

The intricate and overlapping nature of the specific approach taken by the presently described methods therefore provides a test with a much greater level of certainty as relates to an individual patient result, having a much smaller, or even nonexistent, group of patients left without a reliable indicator of risk or direction concerning recommended future treatment.

The late ER+ breast cancer relapse score categorizes a patent in one of two groups based on the expression values of at least eight genes. For example, in one embodiment, the eight genes comprise Homo sapiens ZNF652, PKD1, ZNF786, SPDYE7P, TSC2, ZNF692, DMWD, and MBD4. According to one embodiment of the method, a gene's expression level is assessed using microarray technology where probes to the genes of interest are present on a microarray. In one embodiment, eight microarray probes are utilized to determine the expression level of each of the genes of interest.

In some embodiments, the eight probes are ILMN 2155322, ILMN_2339028, ILMN 1713706, ILMN 1656233, ILMN_1714216, ILMN 1800750, ILMN 1714352, and ILMN 2055310 (these designations are IlluminaHumanv3 probe ID numbers). According to this embodiment, the two groups are low risk and high risk.

In one non-limiting embodiment, density distribution of expression levels from tissues of the heterogeneous patient population is determined based on mixture model fit statistical methods known to those of skill in the art. The key identification, among other things, of a multistate gene threshold specific for a late ER+ breast cancer disease in a human, provides a tool that distinguishes the present disclosure from other work in the human breast disease arts. In addition, the focus on the presence or absence of a particular expression level of a specifically characterized panel of between 8 to 15 genes, from a possible pool of over 20,000 possible gene candidates, imparts a diagnostic and predictive accuracy and robustness to the present techniques that effectively eliminates false negative, false positive and non-conclusive readings for the at risk patient. Because of the bimodal distribution for each gene in the panel, the multistate gene threshold for late breast cancer disease recurrence is used to identify a patient having a late disease occurrence and/or recurrence score falling on one side or the other of the threshold, and thereby identifying the risk of late onset and/or recurrence in the patient having had ER+ breast cancer. The late risk score for the patient is then calculated as the sum of the risk scores for each individual panel gene scaled in a range of 0 to 100.

The present method also provides a set of probes or a set of oligonucleotide primer pairs that comprise detectably labeled single-stranded polynucleotides having specific binding affinity for eight or more genes comprising ZNF652, PKD1, ZNF786, SPDYE7P, TSC2, ZNF692, DMWD, MBD4, HSD17B7, RGS1, GNA11, PHKA2, EGR1, CDC42, and TNRC6A. In another embodiment, the gene panel includes ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1, and ADAM17. In another embodiment, the gene panel includes isoforms of ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1, and ADAM17.

According to another embodiment of the method, the step of determining expression levels of mRNA includes utilizing one or more multi-state probes for the ZNF652, PKD1, ZNF786, SPDYE7P, TSC2, ZNF692, DMWD, and MBD4 gene. According to a further embodiment, the one or more multi-state probes for ZNF652 can be IlluminaHumanv3 probe ILMN_215532; the multi-state probes for PKD1 can be IlluminaHumanv3 probe ILMN_2339028; the multi-state probes for ZNF786 can be IlluminaHumanv3 probe ILMN_1713706; the multi-state probes for SPEDYE7P can be IlluminaHumanv3 probe ILMN_1656233; the multi-state probes for TSC2 can be IlluminaHumanv3 probe ILMN_1714216; the multi-state probes for ZNF692 can be IlluminaHumanv3 probe ILMN_1800750; the multi-state probes for DMWD can be IlluminaHumanv3 probe ILMN_1714352; and the multi-state probes for MBD4 can be IlluminaHumanv3 probe ILMN_2055310. Alternatively, the probes may be mRNA or fragments thereof of the ZNF652, PKD1, ZNF786, SPEDYE7P, TSC2, ZNF692, DMWD, and MBD4 genes or complementary DNA. The probe may be complementary to all or a portion of the mRNA sequence provided that the probe is specific for and can hybridize to the patient's sample under moderately stringent hybridizing conditions, or in another embodiment, stringent hybridization conditions. All of the above recited probes are publicly available.

In yet another aspect, a kit for assessing late onset ER+ breast cancer recurrence in a human at risk patient is provided. The kit comprises a set of detectably labeled probes or a set of oligonucleotide primer pairs having specific binding affinity for at least 8 of the genes comprising: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1 and ADAM17, wherein said detectable label is a non-naturally occurring polynucleotide label. The set of detectably labeled probes or a set of oligonucleotide primer pairs is provided on a solid substrate, and may optionally also include an instructional insert.

### BRIEF DESCRIPTION OF THE DRAWINGS

FIG. 1: According to one aspect of the instant disclosure, the density distribution of the continuous late relapse score in the training set and the validation set is presented. Breast cancer specific deaths (BSD) are indicated (BSD events are indicated in blue and non-events in red). The vertical dotted line separates the late relapse low risk (LateR<31) from the late relapse high risk (LateR>31).

FIG. 2: According to one aspect of the instant disclosure, the Kaplan-Meier plot of the LateR risk groups with a baseline time of 8 years in the validation set is presented. The validation set (n=366) consists of samples in Cohort II that survived at least eight years without BSD event. The Cox proportional hazard model (p=0.03) was calculated with eight years as the baseline time. The late relapse low risk group, indicated in red (LateR<31, 48% of samples), has 20-year BSD-free survival 0.87 (85% CI 0.77-0.97); late relapse high risk group, indicated in blue, has 20-year BSD-free survival 0.70 (85% CI 0.61-0.81).

FIG. 3: According to one aspect of the instant disclosure, the Kaplan-Meier plots of the late relapse risk groups are presented over times from 0 to 20 years Cohort II restricted to (a) LN− and (b) LN+. (a) In LN− the 8-year BSD-free survival probabilities are nearly identical for late relapse low risk, indicated in red (0.902), and late relapse high risk, indicated in blue (0.903), however 20-year BSD-free survival probabilities are markedly different (low risk 0.87 (95% CI 0.80-0.95), high risk 0.70 (95% CI 0.60-0.81). A Cox proportional hazard model over 20 years is not significant (p=0.22) because of the extreme time dependence of the model. (b) In LN+, the risk of relapse is higher in late relapse high risk than in late relapse low risk almost immediately following diagnosis with different 8-year BSD-free survival probabilities, although not statistically significant (low risk 0.74 (95% CI 0.67-0.82), high risk 0.68 (95% CI 0.60-0.77), p=0.17). The 20-year BSD-free survival probabilities are more different (low risk 0.57 (95% CI 0.46-0.72), high risk 0.37 (95% CI 0.24-0.57), and the long-tetni Cox proportional hazard model is significant (p=0.03). Notably, the fraction of late BSD events is significantly higher (p=0.009) in the high-risk group (0.30) than in the low risk group (0.125).

FIG. 4: According to one aspect of the instant disclosure, the Kaplan-Meier plots of the combined early relapse risk group and late relapse risk groups is presented in (a) LN− and (b) LN+ subsets of Cohort II over all times 0 to 20 years. The combination of early relapse and late relapse provides prognosis that is consistently strong over a 20-year span of time. Early relapse gives prognosis from 0 to 8 years and late relapse risk signature predicts relapse from 8 to 20 years. Table 3 details the performance of the combined signature at both early and late time points.

### DETAILED DESCRIPTION

Unless defined otherwise, technical and scientific terms used herein have the same meaning as commonly understood by one of ordinary skill in the art to which the instant disclosure belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The instant disclosure provides a method for predicting the probability of cancer relapse after at least eight years post-diagnosis and the likelihood that a patient will benefit from aggressive chemotherapeutic intervention. The method is based on (1) identifying a panel of gene that correlates with the occurrence of a late ER+ breast cancer disease or recurrence of cancer, (2) determining a risk score for a patient sample, and comparing that risk score to a threshold that stratifies a population of patients into poor prognosis and good prognosis, (3) using that measurement to determine if a patient would benefit from aggressive chemotherapeutic intervention. The method can be used to make treatment decisions concerning the therapy of cancer patients.

One skilled in the art will recognize many methods and materials similar or equivalent to those described herein, which could be used in the practice of the present disclosure. Indeed, the present disclosure is in no way limited to the methods and materials described. For purposes of the present disclosure, the following terms are defined.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

A “gene expression profile” refers to a pattern of expression of at least one biomarker that recurs in multiple samples and reflects a property shared by those samples, such as tissue type, response to a particular treatment, or activation of a particular biological process or pathway in the cells. Furthermore, a gene expression profile differentiates between samples that share that common property and those that do not with better accuracy than would likely be achieved by assigning the samples to the two groups at random. A gene expression profile may be used to predict whether samples of unknown status share that common property or not. Some variation between the levels of at least one biomarker and the typical profile is to be expected, but the overall similarity of the expression levels to the typical profile is such that it is statistically unlikely that the similarity would be observed by chance in samples not sharing the common property that the expression profile reflects.

The term “tag” or “label” is defined as a detectable tag or label, that may be used to detect, monitor, quantify, and otherwise identify the presence or absence of a particular oligonucleotide or specific nucleic acid sequence, and may be used to label or tag a cDNA, cRNA, mRNA, DNA, or any other type of nucleic acid probe or primer. These tags or labels include, by way of example and not limitation, visually detectable labels, such as, e.g., dyes, fluorophores, and radioactive labels, as well as biotin to provide biotinylated species of oligonucleotide, mRNA, cRNA, etc. In addition, the invention contemplates the use of magnetic beads and electron dense substances, such as metals, e.g., gold, as labels. A wide variety of radioactive isotopes may be used including, e.g., 14C, 3H, 99mTc, 123I, 131I, 32P, 192Ir, 103Pd 198AU, 111In, 67Ga, 201TI, 153SM, 18F and 90Sr. Other radioisotopes that may be used include, e.g., thallium-201 or technetium 99m. In other embodiments, the detectable agent is a fluorophore, such as, e.g., fluorescein or rhodamine. A variety of biologically compatible fluorophores are commercially available.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific eDNA (and thus mRNA) of interest.

The term “cRNA” refers to complementary ribonucleic acid, i.e., a synthetic RNA produced by transcription from a specific DNA single stranded template. The cRNA can be labeled with radioactive uracil and then used as a probe. (King & Stansfield, A Dictionary of Genetics, 4th ed.). Alternatively, a non-radioactive label, such as biotin or other non-radioactive label, may be used to label the cRNA probe. cRNA is also described as a single-stranded RNA whose base sequence is complementary to specific DNA sequences (e.g., genes) or, more rarely, another single-stranded RNA, usually conveys an artificial hybridization probe or antisense genetic inhibitor.

As an example, transcriptional activity can be assessed by measuring levels of messenger RNA using a gene chip such as the Affymetrix.RTM. HG-U133-Plus-2 GeneChips. High-throughput, real-time quantitation of RNA of a large number of genes of interest thus becomes possible in a reproducible system.

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

The term “late ER+ breast cancer recurrence” is used in the description of the present invention to mean an ER+ breast cancer that manifests in an ER+ breast cancer patient at least 5 to 20 years after an initial ER+ breast cancer diagnosis.

The term “late ER+-recurrence threshold” as used in the description of the present invention relates to a value that demarcates a high risk late ER+ recurrence group and a low risk late ER+ recurrence group.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression,” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of the instant disclosure, “differential gene expression” is considered to be present when there is at least an about two-fold, preferably at least about four-fold, more preferably at least about six-fold, most preferably at least about ten-fold difference between the expression of a given gene in normal and diseased subjects, or between various stages of disease development in a diseased subject.

The term “prognosis” is used herein to refer to the prediction of the likelihood of cancer-attributable death or progression, including recurrence, metastatic spread, and drug resistance, of a neoplastic disease, such as breast cancer.

The term “prediction” is used herein to refer to the likelihood that a patient will respond either favorably or unfavorably to a drug or set of drugs, and also the extent of those responses; or that a patient will survive, following surgical removal or the primary tumor and/or chemotherapy for a certain period of time without cancer recurrence. The predictive methods of the instant disclosure can be used clinically to make treatment decisions by choosing the most appropriate treatment modalities for any particular patient. The predictive methods of the instant disclosure are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “long-term” survival is used herein to refer to survival for at least 5 years, more preferably for at least 8 years, most preferably for at least 10 years following initial surgery or other treatment.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include, but are not limited to, breast cancer, ovarian cancer, colon cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

In the context of the present invention, reference to “at least eight,” “at least ten,” “at least fifteen,” etc. of the genes listed in any particular gene set means any one or any and all combinations of the genes listed.

The term “node negative” cancer, such as, for example, “node negative” breast cancer, is used herein to refer to cancer that has not spread to the lymph nodes.

The term “sample material” is also designated as a “sample” or as a “specimen” such as a tissue specimen that is fresh frozen, preserved (i.e., FFPE), or otherwise provided in a fresh, preserved or semi-preserved state.

“Biologically homogeneous” refers to the distribution of an identifiable protein, nucleic acid, gene or genes, the expression product(s) of those genes, or any other biologically informative molecule such as a nucleic acid (DNA, RNA, mRNA, iRNA, cDNA etc.), protein, metabolic byproduct, enzyme, mineral etc. of interest that provides a statistically significant identifiable population or populations that may be correlated with an identifiable disease state of interest.

“Low expression,” or “low expression level(s),” “relatively low expression,” or “lower expression level(s)” and synonyms thereof, according to one embodiment of the instant disclosure, refers to expression levels, that based on a mixture model fit of density distribution of expression levels for a particular multi-state gene of interest falls below a threshold “c”, whereas “high expression,” “relatively high,” “high expression level(s)” or “higher expression level(s)” refers to expression levels failing above a threshold “c” in the density distribution. The threshold “c” is the value that separates the two components or modes of the mixture model fit.

The term “gene expression profiling” is used in the broadest sense, and includes methods of quantification of mRNA and/or protein levels in a biological sample.

The term “adjuvant therapy” is generally used to describe treatment that is given in addition to a primary (initial) treatment. In cancer treatment, the term “adjuvant therapy” is used to refer to chemotherapy, hormonal therapy and/or radiation therapy following surgical removal of a tumor, with the primary goal of reducing the risk of cancer recurrence.

“Neoadjuvant therapy” is adjunctive or adjuvant therapy given prior to surgery to remove the tumor. Neoadjuvant therapy includes, for example, chemotherapy, radiation therapy, and hormone therapy. Thus, chemotherapy may be administered prior to surgery to shrink the tumor, so that surgery can be more effective, or, in the case of previously inoperable tumors, possible.

The term “cancer-related biological function” is used herein to refer to a molecular activity that impacts cancer success against the host, including, without limitation, activities regulating cell proliferation, programmed cell death (apoptosis), differentiation, invasion, metastasis, tumor suppression, susceptibility to immune surveillance, angiogenesis, maintenance or acquisition of immortality.

The late relapse score identifies patients at risk for relapse between five and twenty years after diagnosis with ER+ breast cancer, independent of the risk of early relapse (before 5 years), and describes a novel gene expression state of breast cancer tumors (the late relapse high risk group) that exhibit low protein production and other features of a dormant population. Combining the resulting signature with a genomic test for late recurrence of breast cancer provides physicians with a 20-year prognosis to guide long-term treatment decisions. A signature that predicts late recurrence independent of early relapse serves the dual purpose of isolating the biological processes that promote late recurrence and potentially points to more effective treatments.

In one embodiment, the late relapse score comprises expression of a minimum of eight genes to predict the risk of relapse in ER+ breast cancer eight years post-diagnosis. The genes were identified using the Metabric microarray dataset (Curtis et al., 2012) using statistical methods for genomic panel discovery (Bauer, Hummon, & Buechler, 2012; Buechler, 2009). The survival endpoint in the Metabric dataset is breast cancer specific death (BSD).

In another embodiment, a risk score is constructed from gene expression measurements. A gene is considered multistate (Buechler, 2009) if its distribution of expression across a population is sufficiently bimodal, which is formalized with the statistical concept of a mixture model. In building prognostic models, the continuous vector of expression values for a multistate gene is replaced by a binary variable representing the two states, or component groups. As defined herein, the state or component enriched with poor prognosis cases is given the value 1 and the other state or component is given the value 0.

In the instant disclosure, a binary classification variable is replaced with a continuous score that measures the probability of membership in a component; i.e., numbers near 0, 1, or in between, depending on the likelihood that the sample is in the poor prognosis component. This risk score for a gene is calculated by the mixture model methods. The risk score for a gene derived from the mixture model fit in a training set is generalized to a validation set using the statistical method of fitting the same mixture model to the new data.

A prognostic score for a panel of multistate genes is defined as the sum of the risk scores of these genes, resealed to a range of 0-100. This contrasts with the method described by Buechler (Buechler, 2009) in which the multigene prognostic variable is 1 if any of the single-gene variables is 1, and 0 otherwise. Here, samples considered low risk by all of the genes will have a score near 0, and the score increases with the number of genes that classify the sample as high risk.

### EXAMPLES

### Example 1

### Characteristics of Training And Validation Subsets of The ER+ Metabric Microarray Dataset

The present example is provided to define the statistical tools, models and data sets employed to derive the present methods.

All statistical analyses were performed using R (http://www.r-project.org). Mixture models were fit using the package mclust (Fraley & Raftery, 2002; 2012) and survival analysis was performed with the survival package. The significance of a Cox proportional hazard (CPH) model was assessed with the P value of the logrank score test. The significance of a multivariate CPH over a CPH using a subset of the variables was measured with a Chi-squared test of the log-likelihoods. The proportional hazard condition was tested with the cox.zph function.

The Monte Carlo cross-validation (Kuhn & Johnson, 2013) was used to estimate parameters in the development of a predictive model. This method, applied within the training set of model construction, identified models that generalize better than models defined without cross-validation.

The ER+ Metabric dataset (Table 1, (Curtis et al., 2012)) contains gene expression values hybridized to the illuminaHumanv3 array platform. Death due to breast cancer (BSD) is the survival endpoint in this dataset. Cohort I and Cohort II (Table 1) consists of the training and validation cohorts, respectively (Curtis et al., 2012). The training cohort (Cohort I), defined as the sample population with events prior to 8 years excluded (represented by * on Table 1); the validation cohort (Cohort II) defined as the sample population with at least 8 years of BSD-free survival (represented by † on Table 1).

TABLE 1
Characteristics of training and validation subsets of the
ER+ Metabric microarray dataset

| | | | Late | Late |
|---|---|---|---|---|
| | | | Relapse t | Relapse |
| | Cohort I | Cohort II | training | validation |
| | (n = 798) | (n = 720) | (n = 485)* | (n = 366)† |
| Death by breast | 137/48/14 | 109/47/2 | 0/48/0 | 0/47/0 |
| cancer | | | | |
| (time <8 years/ | | | | |
| time ≧8 | | | | |
| years/NA) | | | | |
| LN−/LN+ | 432/366 | 397/323 | 277/208 | 223/143 |
| Grade | 70/392/336/0 | 96/320/234/70 | 53/277/155/0 | 49/163/ |
| (1/2/3/NA) | | | | 111/43 |
| Tamoxifen | 578/220 | 510/210 | 349/136 | 234/132 |
| (yes/no) | | | | |
| Size | 354/444/0 | 315/391/14 | 236/249/0 | 194/164/8 |
| (≦2 cm/>2 cm/ | | | | |
| NA) | | | | |
| Age (<50/≧50) | 143/655 | 104/616 | 97/388 | 64/302 |
| INDUCT | 565/233 | 509/211 | 485/0 | 273/93 |
| (low/high) | | | | |

### Example 2

### Methodology for the Derivation and Validation of the Late Relapse Gene Signature

The following algorithm details the steps used herein. The algorithm was used with Monte Carlo cross-validation to select the parameters n and c, as well as in the ultimate derivation of Late Relapse.
Late relapse training-validation algorithm
An instance of model training and validation is executed with the following
Inputs:
A training set of low INDUCT samples with no relapse events before 8 years;
A validation set with all follow-up times greater than 8 years (hence no relapse events before 8 years), disjoint from the training set;
A number n=the number of genes to use for the panel;
A number c, between 0 and 100, =value of the late relapse score separating the low risk and high risk samples;
A set of multistate genes from which the panel is selected.
Discovery process:
For each candidate multistate variable, the chi-square statistic between the multistate gene's binary variable and the BSD event vector in the training set was computed;
The panel variables P, the genes with the n largest chi-square statistics were selected;
The late relapse score S was formed by adding the individual risk scores of the genes in P and scaling for 0 to 100;
A binary late relapse test T was formed using the value c: the low risk samples were those with S less than c and the high risk samples were those with S greater than or equal to c.
Validation process:
The binary test variable T was computed using a Cox proportional hazard model in the variable T on the assessment set;
The assessment process reported the p-value of the CPH.

### Example 3

### Derivation of the Late Relapse Score and Risk Stratification

The derivation of the late relapse risk stratification required multiple steps to select all of the necessary components. In summary, a panel of multistate genes was selected, a continuous multigene score was constructed, and finally samples were divided into low risk and high-risk groups by comparing the late relapse score value to a threshold value, (c). As detailed in the late relapse Training-Validation Algorithm, the panel of genes was selected as the (n) multistate genes most predictive of late relapse in the training set, for a particular number (n). The execution of the algorithm required first selecting the necessary inputs: (1) training and validation sets, (2) a candidate set of multistate genes, and the numbers (n) and (c).

Samples in the Metabric cohort I (Table 1) were chosen as the training set excluding those with relapse events before 8 years. The restriction in cohort I samples minimized effects of early relapse processes that may have extended beyond eight years. This set consisted of 485 samples with 48 late BSD events. The late relapse validation set consisted of ER+ samples in the Metabric cohort II with follow-up time at least eight years (366 samples with 47 late BSD events).

The pool of multistate genes (i.e., array probes) from which the late relapse gene panel was selected was filtered to exclude probes that (1) were not annotated to a gene and (2) were not contained in a weighted gene coexpression network analysis (WGCNA) module. These restrictions aided the analysis of the biological processes underlying late relapse. In the training step, a multistate gene's level of significance to predict late relapse was measured with the chi-squared statistic of the gene's binary variable and the late relapse event vector. The chi-squared statistic was chosen over a CPH because in the discovery stage there was difficulty with isolating late relapse events (assessed by the chi-squared statistic), while a CPH model gives greater weight to earlier events.

The parameters (n) and (c) required by the algorithm were selected using Monte Carlo cross-validation. A family of 100 training sets, Ti, i <100, were randomly chosen so that each Ti consists of ⅔ of the late relapse training set, for balance. For each i≦100, a validation set, Vi, disjoint from Ti and consisting of ER+ samples in the Metabric cohort I with at least eight years of follow-up was chosen. Note that the Vi's were disjoint from the overall late relapse validation set. Each Ti contained 325 samples with 32 late relapse cases and each Vi contained 124 samples with 17 late events. Candidate values of (n), specifically 5, 10, 15, 20, 30, and candidate values of (c)od, namely integers ranging from 20 to 45, were tested by applying the late relapse derivation algorithm to each pair Ti-Vi, i≦100, and each candidate pair of (n) and (c). From each application the p-values of CPH models were collected and evaluated in Vi for the derived continuous late relapse score and the binary late relapse risk stratification defined using (c). The suitability of the candidate parameters (n) and (c) were assessed using the median p-values ranging over all Ti-Vi, and the median rates of events in the low risk groups.

TABLE 2
Candidate genes for late relapse panel

| | | | WGCNA | High Risk |
|---|---|---|---|---|
| Probe | Symbol | Gene Id | Module | Comp* |
| ILMN_2155322 | ZNF652 | 22834 | 1 | High |
| ILMN_2339028 | PKD1 | 5310 | 13 | High |
| ILMN_1713706 | ZNF786 | 136051 | 1 | High |
| ILMN_1656233 | SPDYE7P | 441251 | 1 | High |
| ILMN_1714216 | TSC2 | 7249 | 13 | High |
| ILMN_1800750 | ZNF692 | 55657 | 1 | High |
| ILMN_1714352 | DMWD | 1762 | 13 | High |
| ILMN_2055310 | MBD4 | 8930 | 11 | High |
| ILMN_1671661 | HSD17B7 | 51478 | 11 | High |
| ILMN_1656011 | RGS1 | 5996 | 12 | Low |
| ILMN_1802397 | GNA11 | 2767 | 13 | High |
| ILMN_1814074 | PHKA2 | 5256 | 1 | High |
| ILMN_1762899 | EGR1 | 1958 | 20 | Low |
| ILMN_1738424 | CDC42 | 998 | 2 | Low |
| ILMN_1714622 | TNRC6A | 27327 | 13 | High |
| ILMN_1757106 | MARCH6 | 10299 | 1 | High |
| ILMN_1701947 | GPR34 | 2857 | 12 | Low |
| ILMN_1778457 | IL18 | 3606 | 1 | High |
| ILMN_2189424 | MRPL20 | 55052 | 3 | Low |
| ILMN_1726809 | BHLHE41 | 79365 | 7 | High |
| ILMN_1669523 | FOS | 2353 | 20 | Low |
| ILMN_2269564 | ARID4B | 51742 | 1 | High |
| ILMN_1755114 | EIF2AK4 | 440275 | 1 | High |
| ILMN_2390472 | TTC14 | 151613 | 1 | High |
| ILMN_1787251 | DAAM1 | 23002 | 1 | High |
| ILMN_2189222 | KLHL8 | 57563 | 1 | High |
| ILMN_2148290 | PDCD7 | 10081 | 1 | High |
| ILMN_1778240 | GFOD1 | 54438 | 1 | High |
| ILMN_1660551 | CRAMP1L | 57585 | 13 | High |
| ILMN_1758392 | ANKS1B | 56899 | 1 | High |
| ILMN_1771962 | GLI3 | 2737 | 2 | High |
| ILMN_2273224 | SLC4A5 | 57835 | 1 | High |
| ILMN_1755990 | ATP6AP1L | 92270 | 1 | High |
| ILMN_1811443 | AVP | 551 | 13 | High |
| ILMN_1702636 | TUBB6 | 84617 | 10 | Low |
| ILMN_2168952 | DENR | 8562 | 1 | High |
| ILMN_1793831 | TRADD | 8717 | 1 | Low |
| ILMN_2342455 | PPA2 | 27068 | 1 | High |
| ILMN_2220320 | RPL7L1 | 285855 | 1 | High |
| ILMN_2121066 | ADAM17 | 6868 | 1 | High |

### Example 4

### Density Distribution of the Continuous Later Score

Assessment of the binary late relapse score risk variables showed that panels using 15 variables performed better than those using fewer variables, but no increase in performance was found with more than 15 variables. For panels with 15 genes, binary tests defined by cuts of 30 to 35 performed equivalently well, with lowest event rates in the low risk groups occurring for cuts 29-33. For these reasons, we chose 15 as the panel size and 31 as the score threshold separating low risk and high risk. The continuous late relapse scores derived in the Ti performed poorly in the Vi in CPH models, so the binary risk stratification was chosen for generalization.

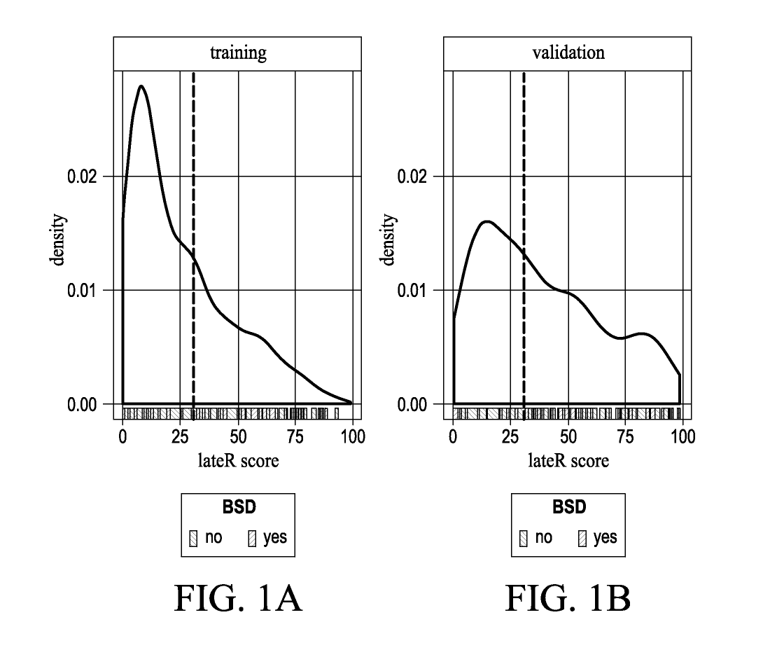

The prioritized set of possible panel of genes (Table 2, ranked by significance) was generated by executing the late relapse training-validation algorithm using the late relapse training set and the multistate candidate probes described above. The fifteen most significant probes were used to define the continuous late relapse score. The late relapse score was extended to the late relapse validation set; the binary late relapse risk stratification was defined using a threshold of 31. The late relapse score had similar distributions in the training and validation sets (FIG. 1).

### Example 5:

### Validation of Late Relapse Prediction Using Later and Long-Term Prognosis Using Induct+Later

The present example is provided to demonstrate the utility of the present assessment tools, late ER+ breast cancer genetic indicator panel, kits, and methods of using these elements, for successfully identifying almost half of the population (48%) at some risk of developing a recurrent form of an ER+ breast cancer, who may successfully opt out of toxic and expensive anti-cancer treatment, without any appreciable increase in mortality. The tools and methods described herein identify 48% of previously positively diagnosed ER+ breast cancer patient survivors, who are at low risk of cancer recurrence after at least 5, 8 or even 20 years of disease-free survival. Patients who have a low risk (LateR) score and are also lymph node (LN) negative have less than and about 0.5% chance of recurrence after 8 years of disease-free survival (Table 3), even with no Tamoxifen or chemotherapy treatment. These patients can be declared “cured” of any recurrent cancer employing the present techniques after 8 years, and thus spared the side effects and expense of treatment. In this way, the present tools and methods may be used to significantly reduce suffering for tens of thousands of women a year.

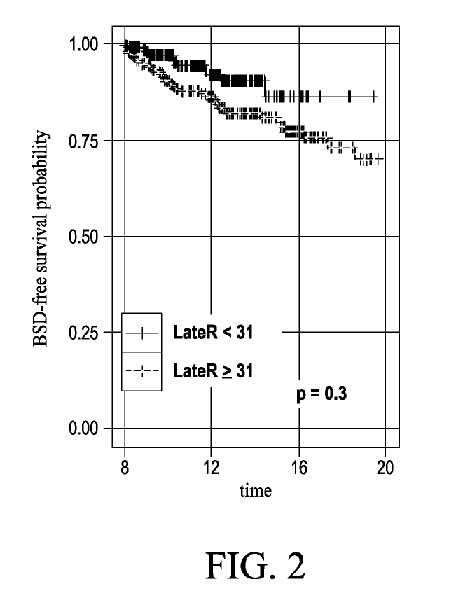

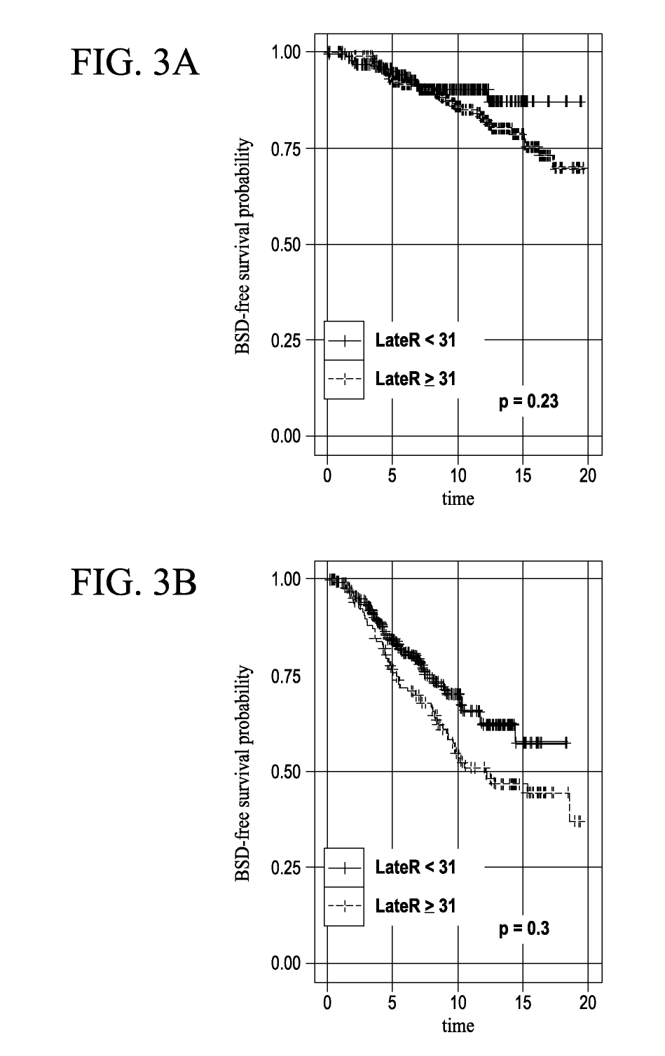

The late relapse score risk stratification (48% low risk, LateR<31) significantly predicts breast cancer specific death events after eight years BSD-free survival in ER+ breast cancer in the validation set (p=0.03, FIG. 2, LateR low risk 20-year BSD-free survival 0.87 (85% CI 0.77-0.97); LateR high risk 20-year BSD-free survival 0.70 (85% CI 0.61-0.81)). The possible effect on disease progression before eight years of the late relapse high-risk factors is best illustrated separately in LN− and LN+ disease (FIG. 3). In LN−, ER+ breast cancer, expected survival probabilities in the late relapse low risk and high risk groups are nearly identical until eight years, at which time they diverge sharply. On the other hand, in LN+, ER+ breast cancer, the patients at high risk for late relapse have poorer prognosis before eight years as well. Notably, late relapse is more prevalent in the high-risk group than in the low risk group in both LN− and LN+ (FIG. 3). The late relapse low risk group contains 47% of LN− samples and 56% of LN+ samples in the validation set.

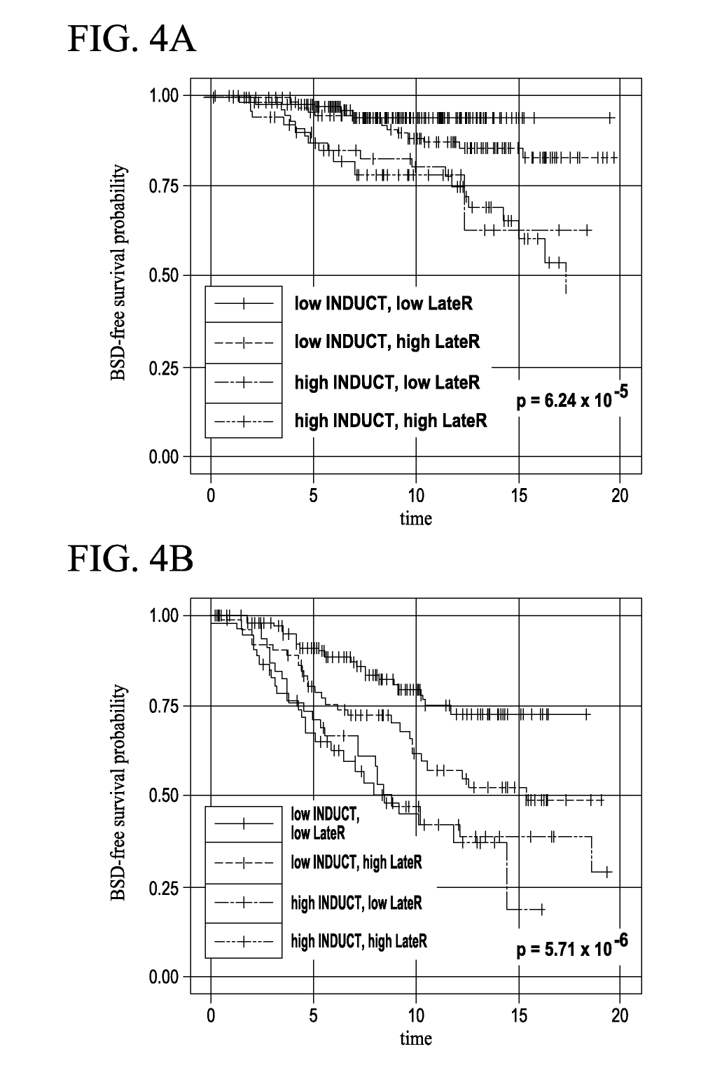

The late relapse score combined with a test to predict the probability of early relapse predicts long-term survival in ER+ breast cancer with consistent significance over 20 years. The stratification of patients into groups that have low or high risk of early relapse and low or high risk of late relapse produces a tool for long-term survival prediction. Expected survival over 20 years for the four strata computed in the validation set, segregated by lymph node status (FIG. 4 and Table 3), shows differential survival characteristics over the full span of years for each of the four groups.

TABLE 3
Survival characteristics of subgroups defined by the combined test for early relapse and
late relapse groups in the Metabric Cohort I.

| | | | | | |
|---|---|---|---|---|---|
| | | | | | 20-Year |
| | | | 8-Year BSD | | BSD-Free |
| | | Early BSD | Free Survival | Late BSD | Survival |
| Risk Group | Size | Events | (95% Cl) | Events | (95% Cl) |

LN− long-term validation set (p = 6.24 × 10⁻⁵)

| | | | | | |
|---|---|---|---|---|---|
| low INDUCT, | 165 | 8 | 0.94 (0.90-0.98) | 0 | 0.94 (0.90-0.98) |
| low LateR | | | | | |
| low INDUCT, | 124 | 7 | 0.94 (0.89-0.98) | 8 | 0.83 (0.75-0.92) |
| high LateR | | | | | |
| high INDUCT, | 51 | 9 | 0.78 (0.67-0.92) | 1 | 0.63 (0.39-1.0) |
| low LateR | | | | | |
| high INDUCT, | 57 | 9 | 0.83 (0.73-0.94) | 9 | 0.45 (0.27-0.74) |
| high LateR | | | | | |

LNs+ long-term validation set (p = 5.71 × 10−6)

| | | | | | |
|---|---|---|---|---|---|
| low INDUCT, | 143 | 18 | 0.84 (0.77-0.91) | 6 | 0.72 (0.63-0.84) |
| low LateR | | | | | |
| low INDUCT, | 77 | 19 | 0.72 (0.62-0.84) | 10 | 0.49 (0.36-0.65) |
| high LateR | | | | | |
| high INDUCT, | 54 | 22 | 0.50 (0.37-0.68) | 4 | 0.19 (0.04-0.80) |
| low LateR | | | | | |
| high INDUCT, | 49 | 17 | 0.61 (0.48-0.78) | 9 | 0.29 (0.14-0.58) |
| high LateR | | | | | |

### Example 6

### Validation of Late Relapse Score as a Predictor of Late Relapse Independent of Clinical Parameters And PAM50

In the validation set of samples with at least eight years of relapse-free survival, LN, tumor grade, PAM50 and INDUCT were found to be significant in univariate CPH models using eight years as the baseline time (Table 4). The late relapse risk signature is significant as a late relapse risk factor in multivariate survival analysis including other risk factors identified above (Table 5), verifying late relapse as an independent test for late relapse risk and supporting the assertion that the different processes drive early and late relapse.

TABLE 4
Significance of clinical variables PAM50 and INDUCT (a test to predict
relapse prior to eight years) as predictors of late relapse in the
validation set

| Variable | p-value |
|---|---|
| Lymph node status (LN+/LN−) | 0.0004 |
| grade | 0.02 |
| Grade (excluding grade 1) | 0.71 |
| Size 2 cm/>2 cm) | 0.33 |
| Age (<50/50) | 0.91 |
| PAM50 | 0.002 |
| INDUCT | 0.0007 |

(p-value of a Cox proportional hazard model with 8 years as a baseline)

TABLE 5
Significance of late relapse signature as a late relapse risk
factor independent of clinical variables, PAM50 and INDUCT
in the validation set. (p value computed using 2-times the
difference of the log-likelihood of a CPH using only the variable
in the first column and a CPH including the variable and the LateR).

| Variable | p-value |
|---|---|
| LN | 0.004 |
| LN + grade | 0.017 |
| LN + PAM50 | 0.003 |
| LN INDUCT | 0.015 |

### Example 7

### Premalignant Lesion and Pre-Invasive Tumor Risk Assessment for Late ER+ Breast Cancer Occurrence

The LateR score predicts recurrence of cancer my measuring gene expression in biopsy tissue that has been confirmed to be ER+ breast cancer. Tissue that is pathologically classified as a pre-malignant lesion, or a pre-invasive tumor, have significant genomic similarity to cancer (Ma et al., 2003). Applied to these pre-cancerous lesions, LateR will predict the onset of invasive breast cancer years hence. (See Ma, X.-J., Salunga, R., Tuggle, J. T., Gaudet, J., Enright, E., McQuary, P., et al. (2003). Gene expression profiles of human breast cancer progression. Proceedings of the National Academy of Sciences of the United States of America, 100(10), 5974-5979. http://doi.org/10.1073/pnas.0931261100).

All of the patents, patent applications, patent application publications and other publications recited herein are hereby incorporated by reference as if set forth in their entirety. The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

1. An assessment tool for late ER+ breast cancer recurrence in an at risk human ER+ breast cancer patient comprising a threshold value that defines a reference heterogeneous late ER+ breast cancer marker of heterogeneous late ER+ breast cancer survivor population gene panel levels, wherein the assessment tool partitions an at risk human ER+ breast cancer tissue score into a high risk or a low risk ER+ breast cancer recurrence group.

2. The assessment tool of claim 1 wherein the heterogeneous late ER+ breast cancer survivor population gene panel comprises at least 8 genes selected from the group consisting of: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1 and ADAM17.

3. The late ER+ breast cancer recurrence assessment tool of claim 1 wherein a low risk human ER+ breast cancer tissue score below an about 60^{th} percentile of the score values in a heterogeneous ER+ breast cancer population indicates a patient with a statistically lower probability of developing late ER+ breast cancer recurrence from 5 to 20 years after an initial ER+ breast cancer occurrence.

4. The late ER+ breast cancer recurrence assessment tool of claim 1 wherein a high risk human ER+ breast cancer tissue score at least above an about 60^{th} percentile or higher of the threshold score values in a heterogeneous ER+ breast cancer population indicates a patient with a statistically higher probability of developing late ER+ breast cancer recurrence from 5 to 20 years after an initial ER+ breast cancer occurrence.

5. The late ER+ breast cancer recurrence assessment tool of claim 1 wherein the level of each gene comprising the heterogeneous late ER+ breast cancer survivor population gene panel is identified with a cDNA, mRNA, cRNA or other nucleotide that is specific for the gene.

6. A set of probes or a set of oligonucleotide primer pairs, wherein each probe or set of oligonucleotide primer pairs is a detectably labeled single-stranded polynucleotide having specific binding affinity for at least 8 of the genes comprising: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1 and ADAM17, wherein said detectable label is a non-naturally occurring polynucleotide label.

7. The set of probes or set of oligonucleotide primer pairs of claim 6 wherein set of probes or set of oligonucleotide primer pairs are provided on a solid substrate.

8. The set of probes or set of oligonucleotide primer pairs of claim 7 wherein the solid substrate is a microchip.

9. A method for determining patient risk for late ER+ breast cancer recurrence comprising:
measuring a patient breast cancer tissue sample from an at risk ER+ breast cancer patient for levels of a heterogeneous late ER+ breast cancer survivor population gene panel comprising at least 8 genes;
calculating a patient gene risk score between 0 and 1 for each gene of the gene panel measured in the patient breast cancer tissue sample;
calculating a patient cumulative cancer test score between 0 to 100 from the patient gene risk score values for each gene of the gene panel; and
comparing said patient cumulative cancer test score to a reference heterogeneous ER+ breast cancer population threshold value;
wherein a patient cumulative cancer test score below about a 60^{th} percentile of the score values in a heterogeneous ER+ breast cancer population indicates a patient with a statistically lower probability of developing late ER+ breast cancer recurrence from 5 to 20 years after an initial ER+ breast cancer occurrence; and
wherein a patient cumulative cancer test score at least above about a 60^{th} percentile or higher of the score values in a heterogeneous ER+ breast cancer population indicates a patient with a statistically higher probability of developing late ER+ breast cancer recurrence from 5 to 20 years after an initial ER+ breast cancer occurrence.

10. The method of claim 9 wherein the patient breast tissue sample is a frozen tissue, formalin fixed, paraffin embedded (FFPE) tissue, or a fresh tissue sample, and the levels of the heterogeneous late ER+ breast cancer survivor population gene panel are provided by measure of a cDNA or cRNA prepared from the patient breast tissue sample.

11. The method of claim 9 wherein an ER+ breast cancer patient having a higher probability of ate ER+ breast cancer recurrence is administered an aggressive anti-cancer therapeutic treatment, and an ER+ breast cancer patient having a lower probability of late ER+ breast cancer recurrence is not administered an aggressive anti-cancer therapeutic treatment.

12. The method of claim 9 wherein the heterogeneous late ER+ breast cancer survivor population gene panel comprises at least 8 genes selected from the group consisting of: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TI C14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1, and ADAM17.

13. The method of claim 9 further comprising the step of
administering an aggressive anti-cancer therapeutic regimen to an ER+ breast cancer patient having a cumulative cancer test score at least within an about 60^{th} percentile or higher of the score values of a reference heterogeneous ER+ breast cancer population , or
not administering an aggressive anti-cancer therapeutic regimen to an ER+ breast cancer patient not demonstrating a cumulative cancer test score at least above an about 60^{th} percentile or higher of the score values of a reference heterogeneous ER+ breast cancer population

14. The method of claim 9 wherein the breast tissue sample is a frozen tissue, a formalin fixed, paraffin-embedded (FFPE) tissue or a fresh tissue sample and the levels of the heterogeneous late ER+ breast cancer survivor population gene panel are provided by measure of a cDNA or cRNA prepared from the patient breast tissue sample.

15. The method of claim 9 wherein the heterogeneous late ER+ breast cancer survivor population gene panel comprises at least 8 genes selected from the group consisting of: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1, and ADAM17.

16. The set of probes of claim 6 comprising a kit for assessing late onset ER+ breast cancer in a human
wherein said probes detectably labeled probes or a set of oligonucleotide primer pairs having specific binding affinity for at least 8 of the genes comprising: ZNF652, PKD1, ZNF786, SPDYE7P, TSC2, ZNF692, DMWD, MBD4, HSD17B7, RGS1, GNA11, PHKA2, EGR1, CDC42, TNRC6A, MARCH6, GPR34, IL18, MRPL20, BHLHE41, FOS, ARID4B, EIF2AK4, TTC14, DAAM1, KLHL8, PDCD7, GFOD1, CRAMP1L, ANKS1B, GLI3, SLC4A5, ATP6AP1L, AVP, TUBB6, DENR, TRADD, PPA2, RPL7L1 and ADAM17, wherein said detectable label is a non-naturally occurring polynucleotide label.

17. The set of probes of claim 16 wherein the set of detectably labeled probes or a set of oligonucleotide primer pairs is provided on a solid substrate.

18. The set of probes of claim 16 wherein said kit further comprises an instructional insert.
